(19) United States
(12) Patent Application Publication
(10) Pub. No.: US 2021/0338773 A1
(43) Pub. Date: Nov. 4, 2021

(54) METHOD FOR TREATING RETINAL DEGENERATION DISEASE BY ADMINISTERING NUCLEOLIN POLYNUCLEOTIDE OR POLYPEPTIDE

(71) Applicants: INSERM (INSTITUT NATIONAL DE LA SANTÉ ET DE LA RECHERCHE MÉDICALE), Paris (FR); SORBONNE UNIVERSITE, Paris (FR); CENTRE NATIONAL DE LA RECHERCHE SCIENTIFIQUE - CNRS -, Paris (FR)

(72) Inventors: Thierry LEVEILLARD, Paris (FR); Najate AIT-ALI, Paris (FR); Frédéric BLOND, Paris (FR)

(21) Appl. No.: 17/282,502

(22) PCT Filed: Oct. 17, 2018
(86) PCT No.: PCT/IB2018/001374

§ 371 Date: Apr. 2, 2021

(51) Int. Cl. A61K 38/17; A61K 35/30; C12N 5/079; C12N 15/86; A61K 9/00

(52) U.S. Cl. CPC A61K 38/1709; A61K 35/30; C12N 2750/14143; C12N 15/86; A61K 9/0048; C12N 5/0621

(57) ABSTRACT

The present invention relates to a new method for treating a patient suffering from a retinal degenerative disease. The Inventors discovered that nucleolin (NCL) is responsible in rods of the production of the short messenger of NXNL1 gene encoding RdCVF, a crucial factor for cones survival. Thus, the administration of NCL into the retina or the overexpression of NCL in recombinant cones to be transplanted into the retina, leads to a RdCVF expression and secretion by the cones themselves in order to encourage their own survival in an autocrine manner through the BSG1/GLUT1 complex. Thus, the invention concerns nucleolin polynucleotide or polypeptide for use in the treatment of a retinal degenerative disease in a patient in need. The invention also relates to recombinant cone overexpressing NCL for use in the treatment of a retinal degenerative disease.

### FIELD OF THE INVENTION

The present invention relates to a method for treating a retinal degenerative disease comprising the administration of a nucleolin polynucleotide or polypeptide.

### BACKGROUND OF THE INVENTION

Photoreceptors are a specialized subset of retinal neurons that are responsible for vision. Photoreceptors consist of rods and cones which are the photosensitive cells of the retina. Each rod and cone elaborates a specialized cilium, referred to as an outer segment that houses the phototransduction machinery. The rods contain a specific light-absorbing visual pigment, rhodopsin, distinct from that of the cones. There are three classes of cones in humans, characterized by the expression of distinct visual pigments: the blue, green and red pigments. Each type of visual pigment protein is tuned to absorb light maximally at specific wavelengths. The rod rhodopsin mediates scotopic vision (in dim light), whereas the cone pigments are responsible for photopic vision (in bright light). The red, blue and green pigments also form the basis of color vision in humans. The visual pigments in rods and cones respond to light and generate an action potential in the output cells, the bipolar neurons, which is then relayed by the retinal ganglion neurons to produce a visual stimulus in the visual cortex.

In human, a number of diseases of the retina involve the progressive degeneration and eventual death of photoreceptors, leading inexorably to blindness. Degeneration of photoreceptors, such as by inherited retinal dystrophies (e.g., retinal degenerative disorders), age related macular degeneration (AMD) and other maculopathies, or retinal detachment, are all characterized by the progressive atrophy and loss of function of photoreceptor.

Retinitis pigmentosa (RP) is a genetically heterogeneous retinal degenerative disease characterized by the progressive death of rod photoreceptors followed by the consecutive loss of cones. RP is one of the most common forms of inherited retinal degeneration, affecting around 2 million people worldwide (Buch et al., 2004). Over 64 mutations causing RP have been identified to date with a significant proportion of these mutations in rod-specific genes. RP patients initially present with loss of vision under dim-light conditions as a result of rod death, with relative preservation of macular cone-mediated vision. As the disease progresses, however, the primary loss of rods is followed by cone degeneration, and a deficit in corresponding cone-mediated vision. In modem society, in which much of the environment is artificially lit, and many activities rely on high acuity color vision, retention of cone-mediated sight in RP patients would lead to a significant improvement in quality of life.

Several international patent applications (WO2010/029130A1, WO2014/060517A1) describes a family of trophic factors, called rod-derived cone viability factor (RdCVF) that are able to increase neuron survival and are useful for treating and/or preventing retinal degenerative disorders such as RP and AMD.

The rod-derived cone viability factor (RdCVF) was originally identified from a high-content method of screening cDNA libraries as a candidate molecule responsible for this rescue effect (Léveillard et al., 2004). Rods secrete RdCVF which helps maintain the cones, and therefore, as rods die, the source of this paracrine factor is lost as RdCVF levels decrease. The loss of expression of RdCVF, and secreted factors like it, may therefore contribute to the secondary wave of cone degeneration observed in retinitis pigmentosa. RdCVF has been shown to mediate cone survival both in culture and when injected subretinally in mouse and rat models of recessive and dominant forms of retinitis pigmentosa (Léveillard et al., 2004; Yang et al., 2009; Byrne et al., 2015).

The nucleoredoxin-like-1 (NXNL1) gene encodes two proteins by an alternative splicing, the short NXNL1 messenger encodes RdCVF that is secreted by rods and protects cones and the long NXNL1 messenger encodes the enzyme RdCVFL (FIG. 1). RdCVFL includes a C-terminal extension conferring enzymatic thioloxidoreductase activity (Brennan et al., 2010) contrary to the short isoform RdCVF mediating cone survival which is a truncated thioredoxin-fold protein. RdCVFL, which contains all the amino acids of RdCVF, is encoded by exons 1 and 2 of the NXNL1 gene and is a member of the thioredoxin-like family (Funato et al., 2007). Thioredoxins have diverse functions, including maintaining the proper reducing environment in cells and regulating apoptotic pathways. These functions are accomplished via thioloxidoreductase reactions mediated by a conserved CXXC catalytic site within a thioredoxin fold (Lillig et al., 2007).

Across the prior art, authors have widely proposed the use of the short and the long isoform encoded by the NXNL1 gene, to treat retinal degenerative disorders.

Byrne et al. (2015) have shown that the two isoforms RdCVF and RdCVFL have complementary functions. Systemic administration of an adeno-associated virus (AAV) encoding RdCVF improved cone function and delayed cone loss, while RdCVFL increased rhodopsin mRNA and reduced oxidative stress. RdCVFL prevents photo-oxidative damage to the rods (Elachouri et al., 2015).

International patent application WO2016/185037 describes AAV vectors encoding both RdCVF and RdCVFL, and the use of said vectors for treating pathologies such as RP.

A synergistic effect between RdCVF and RdCVFL has been postulated (Mei et al., 2016). On the one hand, RdCVF is produced and secreted by the rods, and stimulates the renewal of cones outer segments by stimulating aerobic glycolysis though the RdCVF receptor, Basigin-1 (BSG1), at the cell surface of the cones (Aït-Ali et al., 2015). When RdCVF binds to BSG1, a transmembrane protein with three immunoglobulin-like domains in its extracellular portion, it activates the glucose transporter GLUT1 (SLC2A1), resulting in increased glucose entry into cones. Increased glucose promotes cone survival by stimulation of aerobic glycolysis. On the other hand, RdCVFL protects the cones against oxidative damage in a cell autonomous manner, due to its thioloxidoreductase activity that relies on the metabolism of glucose through the pentose phosphate pathway (Léveillard et al., 2017).

The alternative splicing leading to intron retention that results in the production of the RdCVF mRNA from the NXNL1 gene which takes place specifically in rods, remained unknown.

Besides, nucleolin (NCL) is a protein expressed by many cell types in organisms. It was firstly identified in the nucleolus, wherein NCL is known to interact with certain RNA helicases, enzymes that catalyze the opening of a nucleic acid chain. NCL is a protein also known to be involved in various important steps of the cellular process such as chromatin remodeling, DNA replication and recombination, regulation of gene expression, RNA metabolism, response stress, proliferation and cell growth, transcription, cellular signal transduction, RNA splicing and ribosome biogenesis. It is also known that NCL can interact with many proteins and that many of its partners can participate in the different steps of pre-mRNA metabolism by transcribing them to their export to the cytoplasm (Salvetti et al., 2016; Shin et al., 2018).

### SUMMARY OF THE INVENTION

The Inventors have surprisingly discovered that NCL plays a role in promoting NXNL1 intron retention. They showed that NCL promotes the production of the short messenger from NXNL1 gene encoding RdCVF by rods. Thus, promoting the alternative splicing in cones as it normally occurs in rods, by overexpressing NCL in cones leads to the RdCVF expression and secretion in order to encourage cone survival in an autocrine manner through the BSG1/GLUT1 complex.

Unless otherwise specified, the term “RdCVF” refers to the polypeptide encoded by the short messenger of the NXNL1 gene and “RdCVFL” refers to the polypeptide encoded by the long messenger of the NXNL1 gene.

The Inventors have identified a conserved stem loop in the NXNL1 pre-mRNA which specifically binds NCL, leading therefore to the NXNL1 intron retention. The Inventors have therefore renamed this loop ‘nucleolin responsive element’ (NRE).

Based on this observation, the Inventors have proposed a novel method for treating a retinal degeneration disease, in particular to treat photoreceptors degeneration, based on the use of nucleolin polynucleotide or polypeptide.

Hence, the present invention relates to a method for treating a patient suffering from a retinal degenerative disease comprising a step consisting of administering to said patient a therapeutically effective amount of nucleolin polynucleotide or polypeptide.

In a particular embodiment, the present invention relates to a method for treating a patient suffering from a retinal degenerative disease comprising a step consisting of administering to said patient a therapeutically effective amount of NCL polynucleotide or polypeptide, wherein said patient has been transplanted with cones.

The present invention also relates to an adeno-associated vector (AAV) comprising an expression cassette comprising a polynucleotide encoding the NCL, in particular said polynucleotide has the sequence corresponding of the accession number on NCBI GeneID 4691.

### DETAILED DESCRIPTION OF THE INVENTION

The aim of the present invention is to propose a new method for treating a patient suffering from a retinal degenerative disease.

The present invention relates to a method for treating a patient suffering from a retinal degenerative disease comprising a step consisting of administering to said patient a therapeutically effective amount of nucleolin polynucleotide or polypeptide.

A retinal degenerative disease is a disease presenting photoreceptors degeneration such as cones and/or rods degeneration. As the rods are essential to the cones survival, thanks to the production of RdCVF as explained above, the degeneration of rods leads to the degeneration of cones. Thus, the degeneration of cones is linked to the degeneration of rods. Moreover, as the cones are responsible of the photopic vision it is crucial to prevent and treat their degeneration.

In a particular embodiment wherein the patient suffering from a retinal degenerative disease is at an earlier stage of the disease which means that despite the degeneration of the rods, rods are still present in retinas in sufficient quantity to maintain cones, treating the retinal degenerative disease by administering NCL consists in preventing the degeneration of the cones. In this earlier stage, the degeneration of cones did not start, thus NCL has a preventive effect on the cones degeneration.

In a particular embodiment wherein the patient suffering from a retinal degenerative disease is at an advanced stage of the disease which means that cones degeneration has already started, treating the retinal degenerative disease by administering NCL consists in treating the disease by promoting the survival of the remaining cones.

Thus, in one embodiment, the present invention relates to a method for preventing or treating cones degeneration in a patient suffering from a retinal degenerative disease comprising a step consisting of administering to said patient a therapeutically effective amount of NCL polynucleotide or polypeptide.

According to the present application, the retinal degenerative disease is selected in the group consisting of retinitis pigmentosa (RP), Leber congenital amaurosis (LCA), age-related macular degeneration (AMD), recessive RP, dominant RP, X-linked RP, incomplete X-linked RP, dominant, dominant LCA, recessive ataxia, posterior column with RP, recessive RP with para-arteriolar preservation of the RPE, RP 12, Usher syndrome, dominant retinitis pigmentosa with sensorineural deafness, recessive retinitis punctata albescens, recessive Alström syndrome, recessive Bardet-Biedl syndrome, dominant spinocerebellar, ataxia w/macular dystrophy or retinal degeneration, Recessive abetalipoproteinemia, recessive retinitis pigmentosa with macular degeneration, recessive Refsum disease adult form, recessive Refsum disease infantile form, recessive enhanced S-cone syndrome, RP with mental retardation, RP with myopathy, recessive Newfoundland rod-cone dystrophy, RetRP sinpigmento, sector RP, regional RP, Senior-Loken syndrome, Joubert syndrome, Stargardt disease juvenile, Stargardt disease late onset, dominant macular dystrophy Stargardt type, dominant Stargardt-like macular dystrophy, recessive macular dystrophy, recessive fundus flavimaculatus, recessive cone-rod dystrophy, X-linked progressive cone-rod dystrophy, dominant cone-rod dystrophy, cone-rod dystrophy; de Grouchy syndrome, dominant cone dystrophy, X-linked cone dystrophy, recessive cone dystrophy, recessive cone dystrophy with supernormal rod electroretinogram, X-linked atrophic macular dystrophy, X-linked retinoschisis, dominant macular dystrophy, dominant radial, macular drusen, dominant macular dystrophy, bull's-eye, dominant macular dystrophy butterfly-shaped, dominant adult vitelliform macular dystrophy, dominant macular dystrophy North Carolina type, dominant retinal-cone dystrophy 1, dominant macular dystrophy cystoid, dominant macular dystrophy, atypical vitelliform, foveomacular atrophy, dominant macular dystrophy Best type, dominant macular dystrophy North Carolina-like with progressive, recessive macular dystrophy juvenile with hypotrichosis, recessive foveal hypoplasia and anterior segment dysgenesis, recessive delayed cone adaptation, macular dystrophy in blue cone monochromacy, macular pattern dystrophy with type II diabetes and deafness, Flecked retina of Kandori, pattern dystrophy, dominant Stickler syndrome, dominant Marshall syndrome, dominant vitreoretinal degeneration, dominant familial exudative vitreoretinopathy, dominant vitreoretinochoroidopathy; dominant neovascular inflammatory vitreoretinopathy, Goldmann-Favre syndrome, recessive achromatopsia, dominant tritanopia, recessive rod monochromacy, congenital red-green deficiency, deuteranopia, protanopia, deuteranomaly, protanomaly, recessive Oguchi disease, dominant macular dystrophy late onset, recessive gyrate atrophy, dominant atrophia areata, dominant central areolar choroidal dystrophy, X-linked choroideremia, choroidal atrophy, central areolar, central, peripapillary, dominant progressive bifocal chorioretinal atrophy, progressive bifocal choroioretinal atrophy, dominant Doyne honeycomb retinal degeneration (Malattia Leventinese), amelogenesis imperfecta, recessive Bietti crystalline corneoretinal dystrophy, dominant hereditary vascular retinopathy with Raynaud phenomenon and migraine, dominant Wagner disease and erosive vitreoretinopathy, recessive microphthalmos and retinal disease syndrome; recessive nanophthalmos, recessive retardation, spasticity and retinal degeneration, recessive Bothnia dystrophy, recessive pseudoxanthoma elasticum, dominant pseudoxanthoma elasticum; recessive Batten disease (ceroid-lipofuscinosis), juvenile, dominant Alagille syndrome, McKusick-Kaufman syndrome, hypoprebetalipoproteinemia, acanthocytosis, palladial degeneration; Recessive Hallervorden-Spatz syndrome; dominant Sorsby's fundus dystrophy, Oregon eye disease, Kearns-Sayre syndrome, RP with developmental and neurological abnormalities, Basseb Korenzweig Syndrome, Hurler disease, Sanfilippo disease, Scieie disease, melanoma associated retinopathy, Sheen retinal dystrophy, Duchenne macular dystrophy, Becker macular dystrophy, Birdshot Retinochoroidopathy, multiple evanescent white-dot syndrome, acute zonal occult outer retinopathy, retinal vein occlusion, retinal artery occlusion, diabetic retinopathy, retinal toxicity, retinal injury, retinal traumata and retinal laser lesions, and Fundus Albipunctata, retinal detachment, diabetic retinopathy, retinopathy of prematurity.

In particular, according to the present application, the retinal degenerative disease is selected in the group consisting of retinitis pigmentosa, age-related macular degeneration, Bardet-Biedel syndrome, Bassen-Kornzweig syndrome, Best disease, choroidema, gyrate atrophy, Leber congenital amaurosis, Refsum disease, Stargardt disease and Usher syndrome.

More particularly, according to the present application, the retinal degenerative disease is the retinitis pigmentosa.

More particularly, according to the present application, the retinal degenerative disease is the retinitis pigmentosa.

In the context of the invention, the term “treating” or “treatment”, as used herein, means reversing, alleviating, inhibiting the progress of, or preventing the disorder or condition to which such term applies, or one or more symptoms of such disorder or condition (e.g., retinal degenerative diseases).

According to the invention, the term “patient” or “patient in need thereof” is intended for a human affected or likely to be affected with a retinal degenerative disease.

By a “therapeutically effective amount” of the polynucleotide or polypeptide of the invention is meant a sufficient amount of the polypeptide or the polynucleotide to achieve a desired biological effect, in this case treating retinal degenerative disease at a reasonable benefit/risk ratio applicable to any medical treatment. The specific therapeutically effective dose level for any particular subject will depend upon a variety of factors including the disorder being treated and the severity of the disorder; activity of the specific polypeptide employed; the specific composition employed, the age, body weight, general health, sex and diet of the subject; the time of administration, route of administration, and rate of excretion of the specific polypeptide employed; the duration of the treatment; drugs used in combination or coincidental with the specific polypeptide employed; and like factors well known in the medical arts. However, the preferred dosage can be tailored to the individual subject, as is understood and determinable by one of skill in the art, without undue experimentation.

In a particular embodiment, the NCL polypeptide corresponds to the human nucleolin polypeptide, more particularly to the polypeptide corresponding to the Uniprot number P19338.

In one embodiment, the nucleolin polypeptide corresponds to a fragment of the human NCL polypeptide, said fragment having the property to bind to the NRE sequence of SEQ ID NO:1.

In one embodiment, the nucleolin polypeptide corresponds to a fragment of the human NCL polypeptide, said fragment comprising a binding domain to the NRE sequence of SEQ ID NO: 1.

In a particular embodiment, the NCL polynucleotide corresponds to the human NCL polynucleotide. Particularly, the NCL polynucleotide corresponds to the polynucleotide encoding the human NCL polypeptide of Uniprot number P19338. More particularly, the NCL polynucleotide corresponds to the polynucleotide of accession number on NCBI GeneID 4691.

In a particular embodiment, the NCL polynucleotide corresponds to a fragment of the human NCL polynucleotide, said fragment of the NCL polypeptide having the property to bind to the NRE sequence of SEQ ID NO: 1.

In a particular embodiment, the NCL polynucleotide corresponds to a fragment of the human NCL polynucleotide, said fragment of the NCL comprising a binding domain to the NRE sequence of SEQ ID NO: 1.

In a particular embodiment, the treatment with NCL polynucleotide is performed by gene therapy.

In one embodiment, the NCL polynucleotide is comprised in an expression vector.

As used herein, the term “expression vector” refers to a nucleic acid molecule capable of directing the expression of genes to which they are operably linked. One type of expression vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses (AAV)), which serve equivalent functions.

In a particular embodiment, said expression vector is selected from the group consisting of plasmids and viral particles.

In particular, the expression vector comprises suitable promoter enabling the expression in the retina, preferably in cone photoreceptors cells.

In one embodiment, the expression vector comprises a cone specific promoter. A non-limiting example is the cone-opsin promoter.

In a particular embodiment, the vector is an adeno-associated vector (AAV).

As used herein, the term «adeno-associated vector» or «AAV» has its general meaning in the art.

Adeno-associated viral (AAV) vectors have become powerful gene delivery tools for the treatment of retinal degeneration. AAV vectors possess a number of features that render them ideally suited for retinal gene therapy, including a lack of pathogenicity, minimal immunogenicity, and the ability to transduce postmitotic cells in a stable and efficient manner. In the sheltered environment of the retina, AAV vectors are able to maintain high levels of transgene expression in the retinal pigmented epithelium (RPE), photoreceptors, or ganglion cells for long periods of time after a single treatment. Each cell type can be specifically targeted by choosing the appropriate combination of AAV serotype, promoter, and intraocular injection site.

Typically, AAVs according to the present invention are AAVs that are able to target retinal cells. Examples include, but are not limited to AAV2, AAV2/8, AAV8, AAV9 and AAV7m8.

In one embodiment, the AAV according to the present invention is obtained according to the method described in the international patent application WO2012/158757.

In a particular embodiment, the NCL polynucleotide or polypeptide is suitable for intraocular administration. The NCL polynucleotide or polypeptide is administered into the retina, more particularly into the macula. In a particular embodiment, the NCL polynucleotide or polypeptide is administered by sub-retinal injection or intravitreal injection to the patient.

In particular, the NCL polynucleotide or polypeptide is administered into the retina at the level of the macula.

In a particular embodiment, the NCL polynucleotide or polypeptide is formulated in a pharmaceutically acceptable ophthalmic vehicle.

In a particular embodiment, the NCL polynucleotide or polypeptide is formulated in a hydrogel.

Particularly, said hydrogel is a hydrogel comprising a blend of hyaluronan and methylcellulose (HAMC).

HAMC leverages the shear thinning nature of hyaluronan and the inverse thermal gelling properties of methylcellulose to yield a hydrogel that can be injected through a fine needle and rapidly gels in vivo.

In a particular embodiment, the NCL polynucleotide or polypeptide is administered by intravascular injection as described in the application WO2016185037A1.

The present invention also relates to a NCL polynucleotide or polypeptide for use in the treatment of a retinal degenerative disease. All the embodiments described in the present application for a method for treating a retinal degenerative disease apply to this use.

The present invention also relates to an adeno-associated vector (AAV) comprising an expression cassette comprising a polynucleotide encoding the nucleolin.

In a particular embodiment, said AAV comprises an expression cassette comprising a polynucleotide encoding the human NCL polypeptide.

In particular, said AAV comprises an expression cassette comprising a polynucleotide encoded the human NCL polypeptide of Uniprot number P19338.

In particular, said AAV comprises an expression cassette comprising the human NCL polynucleotide of accession number on NCBI GeneID 4691.

Typically, in patients suffering from a retinal degenerative disease, in particular suffering from retinitis pigmentosa or AMD, cones can be transplanted into the retina at the level of the macula.

In particular, the cones which are transplanted come from the in vitro differentiation of induced pluripotent stem cells (iPSC). In particular, the cones are obtained by iPSC differentiation by a method such as the one described in Decembrini et al. (2017).

After transplantation into the retina, more particularly into the macula of the retina, cones survival depends of rods, and more particularly to a factor secreted by rods (RdCVF). Thus, in a patient suffering from a retinal degenerative disease it is still challenging to maintain cones in an environment without rods and to prevent the transplanted cones from degeneration. Thus, the use of NCL in case of cones transplantation can overcome this problem.

In a particular embodiment, the present invention relates to a method for treating a patient suffering from a retinal degenerative disease comprising a step consisting of administering to said patient a therapeutically effective amount of NCL polynucleotide or polypeptide as described above, wherein said patient has been transplanted with cones.

In a particular embodiment, the NCL polynucleotide or polypeptide is administered before, in the same time or after the transplantation of cones.

In a particular embodiment, the transplanted cones are obtaining from in vitro differentiation of iPSC. They are then called induced pluripotent stem cell-derived cones (iPSC-cones).

In another embodiment, the transplanted cones can be genetically engineered to overexpress NCL. These recombinant cones overexpressing NCL are therefore able to have a RdCVF expression and secretion in order to encourage their own survival in an autocrine manner through the BSG1/GLUT1 complex. The recombinant cones overexpressing NCL can survive without rods after their transplantation into the retina.

Thus, the present invention also relates to recombinant cones overexpressing NCL. In a particular embodiment, the recombinant cones overexpressing NCL are obtained by transforming cones cells with an expression vector comprising a polynucleotide encoding NCL. More particularly, said expression vector is an AAV such as those previously described. In particular, the recombinant cones overexpressing NCL are iPSC-cones.

The present invention also relates to a method for treating a patient suffering from a retinal degenerative disease comprising a step of transplanting recombinant cones overexpressing NCL into the retina of the patient, more particularly into the macula. In particular the recombinant cones overexpressing NCL are recombinant iPSC-cones overexpressing NCL.

The present invention also relates to a recombinant cone overexpressing NCL for use in the treatment of a retinal degenerative disease in a patient in need.

In a particular embodiment, the present invention relates to a recombinant cone overexpressing NCL for use as described, wherein the recombinant cone overexpressing NCL is transplanted into the patient retina.

### FIGURES

FIG. 1: Intron retention of the NXNL1 gene produces RdCVF.

FIG. 2: Representative diagram of isoforms of the NXNL1 gene. A) Schematic representation of the NXNL1 gene. B) Schematic representation of the short messenger encoded by NXNL1 gene. C) Schematic representation of the long messenger encoded by NXNL1 gene. The exons are represented by rectangles and the introns by a line connecting the exons. Arrows symbolize primers for RT-PCR.

FIG. 3: Expression of NXNL1 gene products in different parts of macaque retina. A) Quantitative RT-PCR of short NXNL1 mRNA expression in fovea, macula, near peripheral retina, medium peripheral retina and distant peripheral retina. B) Quantitative RT-PCR of long NXNL1 mRNA expression in fovea, macula, near peripheral retina, medium peripheral retina and distant peripheral retina. u.a.: arbitrary unit.

FIG. 4: Expression of NXNL1 gene products in different parts of human retina. A) Quantitative RT-PCR of short NXNL1 mRNA expression in fovea, macula, near peripheral retina, medium peripheral retina and distant peripheral retina. B) Quantitative RT-PCR of long NXNL1 mRNA expression in fovea, macula, near peripheral retina, medium peripheral retina and distant peripheral retina. u.a.: arbitrary unit.

FIG. 5: Phylogenic conservation of the NRE.

FIG. 6: Sequence and secondary structure of NRE (A) and NRE shuffle (B) obtained with mfold Web Server software.

FIG. 7: Specific interaction between the NRE sequence and the nucleolin protein (NCL). A) Image of the HuProt chip region where NCL is located after incubation with the NRE sequence. B) Image of the HuProt chip region where NCL is located after incubation with the NRE shuffle sequence.

FIG. 8: Expression of the mRNA of NCL in the different parts of macaque retina. u.a.: arbitrary unit.

FIG. 9: Quantification of NCL full-length and NCL-fragment expression obtained by western blot, in the different parts of macaque retina.

FIG. 10: Schematic representation of the alternative splicing in presence of NCL.

FIG. 11: A) Density of cones and rods according to the retinal eccentricity (0° represents the fovea) in the different parts of macaque retina: 1: fovea/macula, 2: near peripheral retina, 3: medium peripheral retina, 4: distant peripheral retina. B) Expression of NCL full-length (100 kDa) and NCL-fragment (60 kDa) in the different parts of macaque retina. C) Expression of RdCVF mRNA (short messenger encoded by NXNL1 gene) in the different parts of macaque retina.

FIG. 12: Gel-shift assay performed from incubation of 3, 10 or 80 μg of protein extract of HEK293 cells with radiolabeled NRE or NRE shuffle probe. Negative control (0) consists of radiolabeled NRE probe incubated with water.

### EXAMPLES

Material and Methods

Retina Dissection

The macaque or human eyeballs are placed in a Petri dish with PBS so that the retina does not dry. They are then washed twice in washing solution and once in an independent CO₂ medium (ThermoFisher). One of the eyeballs is pierced with a needle to cut and remove the cornea using a pair of scissors and forceps. The choroid is removed and the sclera delicately detached from the optic nerve using a pair of scissors. The retinal pigmented epithelium (RPE) and vitreous are gradually detached from the retina in small cuts so as not to damage or tear. At this point, the retina has retained its curved shape and needs to be flattened. But before that, the vitreous humor, which has the consistency of a jelly, must be removed in one piece. To flatten the retina, several radial cuts are made using a scalpel blade. Next, fovea, macula, near peripheral retina, medium peripheral retina and distant peripheral retina were isolated.

Concerning the macaque retinas, the eyeballs of a macaque (Macaca fascicularis) used in other experiments and from the platform of MIRCen (Molecular Imaging Research Center) located at the CEA (Commission for Atomic Energy) of Fontenay-aux-Roses have been recovered. Concerning the human retinas, eyeballs come from human organ donor.

RNA Extraction

Total RNA is extracted from each part of the macaque or human retina. RNA extraction is performed using the RNAeasy Plus micro kit (Qiagen) according to the manufacturer's recommendations. 300 μl of lysis buffer RLT is added to each eppendorf tube in which the different parts of the retina were collected during the dissection. Tissues are homogenized using the Kimematica PT2100 polytron. Centrifugation is performed for 2 min at 13,000 rotations per minute (rpm). The lysate is recovered and filtered through a gDNA eliminator column by centrifugation for 2 min at 13,000 rpm before being loaded onto a RNAeasy mini column. Then, three washes are carried out, and the RNAs are eluted in 50 μl of TE (Tris-HCl pH 7.5, 0.1 mM EDTA). Their concentrations are measured using the NanoDrop® ND-1000 spectrophotometer (Labtech).

Reverse Transcription

Complementary DNA synthesis (cDNA) is performed using a reverse transcriptase (RNA dependent-DNA polymerase) Superscript II RNase H-Reverse Transcriptase (Invitrogen), a random sequence hexamer Random Primers (Promega), and from 1 μg of RNA. The cDNA is purified by phenol/chloroform (25:24:1, v/v), precipitated with ethanol and dissolved in 50 μl of Tris-HCl 10 mM, pH 8.0; EDTA 1 mM. A negative reverse transcription control is performed with water in place of the RNA and another without reverse transcriptase.

RT-PCR

The sequences of the specific oligonucleotide primers were designed using the Primer3-Input software (http://primer3.ut.ee/) and synthesized at the commercial provider Life Technologies. Three sequential cDNA dilutions were made and triplicated on a 96-well plate (qPCR 96-well plate, Roche), as well as a negative control (without cDNA). Ten μl of the reagent (qPCR MasterMix) containing the DNA polymerase (Taq DNA polymerase, Roche) and the SYBR Green fluorophore (a DNA intercalant), as well as 4 μM forward primer and 4 μM reverse primer are deposited in a 96-well plate. Eight μl of the diluted cDNA sample is added to this solution. The plate is covered with a transparent film and centrifuged for 1 min at 900 rpm. The amplification and reading of the fluorescence are performed on a thermal cycler (7500 real time PCR System, Applied Biosystems). The PCR cycles consist of a first denaturation of the DNA polymerase for 5 min at 95° C. Then 40 cycles of: denaturation of the DNA for 15 sec at 95° C. followed by primers pairing for 20 sec at 60° C. and finally synthesis of the complementary strand for 30 sec at 72° C. This reaction was then terminated by DNA denaturation for 1 min at 95° C., followed by renaturation of the DNA for 3 sec at 55° C. and a further denaturation of the DNA for 30 sec. at 95° C. Fluorescence is read at the end of each pairing step.

The measurement of gene expression by quantitative RT-PCR (qPCR) requires the use of a gene whose expression does not vary or very little. For this, the gene encoding actin, a quasi-ubiquitous cytoskeletal protein that is highly conserved through the evolution, or the gene that codes for glyceraldehyde-3-phosphate dehydrogenase (GAPDH), which is a ubiquitous enzyme catalyzing the sixth step of glycolysis, are used.

RT-PCR Primers

The following primers (Table 1) are used to carry out the RT-PCR assays.

TABLE 1
Primers used for RT-PCR

| | | | |
|---|---|---|---|
| FIGS. | SHORT | Forward primer | SEQ ID NO: 3 |
| 3, 4 | NXNL1 | hRdCVF F Exon 1 | GAGTTCTATGTACTGCGGGC |
| and | | Reverse primer | SEQ ID NO: 4 |
| 11C | | hRdCVF R | CTTCACTTTCAGCGAACATGC |
| | | Intron 1 | |
| | LONG | Forward primer | SEQ ID NO: 3 |
| | NXNL1 | hRdCVF F Exon 1 | GAGTTCTATGTACTGCGGGC |
| | | Reverse primer | SEQ ID NO: 5 |
| | | hRdCVF R Exon 2 | TCCACTGAGAACTGGCGC |
| FIG. 8 | NCL | Forward primer | SEQ ID NO: 6 |
| | mRNA | hNCL 1 F | GCGTTGGAACTCACTGGTTT |
| | | Reverse primer | SEQ ID NO: 7 |
| | | hNCL 1 R | CCGCAGCATCTTCAAACACT |

Western Blot

Western blotting allows to demonstrate the expression of a protein in a cell extract after migration by the use of antibodies. For each cell extract, 200 μl of lysis buffer [50 mM Tris-HCl; 10 mM EDTA; dithiothreitol (DTT) 1 μM], then 15 μl of protease inhibitors 100×, 150 μl of Triton X-100 10% and 75 μl of tosyl Lys chloromethyl ketone (TLCK) 1 mg/μl. The samples are homogenized and centrifuged for 5 min at 14,000 rpm. The mass concentration of protein is evaluated by the Bradford method. Forty μg of protein are separated on a polyacrylamide gel NuPAGE® Novex 4-12% Bis-Tris (Invitrogen) in a buffer NuPAGE® MES SDS Running Buffer (Invitrogen) at 180 V, and then transferred to a 0.2 μm nitrocellulose membrane (GE Healthcare), for 2 h at 60 V, using a buffer with 25 mM Tris-base; 200 mM glycine; 20% ethanol. The non-specific sites are saturated in a solution of phosphate buffered saline (PBS) comprising blocker 5%; Tween-20 0.05%, for 1 h. Then the membrane is incubated in the presence of the primary antibodies in a solution of PBS comprising Blocker 3%, Tween-20 0.05%, for 3 hours at room temperature (or overnight at +4° C.). After 3 washing for 15 minutes with a solution of PBS with Tween-20 0.05%, the membrane is incubated again for 1 hour at room temperature with the secondary antibody in a solution of PBS with Blocker 3%; Tween-20 0.05%. The revelation is performed by the kit ECL Plus™ Western Blotting Detection Systems (GE Healthcare). Molecular weights of proteins are estimated using the molecular weight standard Kaleidoscope Polypeptide Standards (Bio-rad).

The primary antibodies used are:
Antibody anti Rhodopsin (Millipore: MAB5316) 1/150
Antibody anti GNAT2 (generous gift of James Hurley) 1/100
Antibody anti NCL (Abcam: 22758) 1 μg/ml
Antibody anti GAPDH (Abcam: 9485) 1/2500

Computer Modeling of NRE Secondary Structure

The RNAfold web server software is used to predict secondary structures of single RNA strands. http://rna.tbi.univie.ac.at/cgi-bin/RNAfold.cgi. The software called The mfold Web Server is used to establish the strength and stability of secondary structures of single strands of RNA http://mfold.rna.albany.edu/?q=mfold/RNA-Folding-Form.

Hybridization Test of the NRE and the NRE Shuffle RNA Probes on HuProt™ Human Proteome Microarray

For the test involving the HuProt™ Human Proteome Microarray chip, all the reactions take place in a very cold and dry environment, the chip is stored at −80° C. and then placed on ice the time of the experiment. Each chip is placed in a well of a 4-wells Nunc™ Rectangular Dishes plate (ThermoFisher). 3 ml of blocking solution at 2% SAB/TBS-T (Tris pH 7.5 and 0.1% Tween-20) is added per well. The chips are incubated in this solution at room temperature for 2 h with gentle agitation. The chips are then removed from the wells, tapped on absorbent paper to remove the excess from the blocking solution and placed in a humid chamber to prevent evaporation. The NRE and NRE Shuffle probes are deposited on the surface of the chips, then they are covered with a glass slide and the wet chamber is covered with aluminum foil to limit exposure to light. The whole is stirred for 1 h at room temperature. The incubation solution is replaced by 4 ml PBS-Tween-20 1/1000. This washing step is repeated 3 times, and then 4 ml of this washing solution are added and incubated for 10 min. This last washing step is repeated a second time. Then, the chip is washed 4 times with demineralized water. The water is removed and the chip is dried by tapping on paper absorbent and then centrifuged for 3 minutes at 800 rpm. Finally, the chip is scanned and analyzed by The GenePix® 4000B Microarray Scanner.

Gel-Shift Assay

Proteins are extracted from HEK293 cells. The protein extract is then incubated with radiolabeled probes NRE or NRE shuffle. Negative control consists of NRE probe incubated with water. Three protein extracts quantities are performed: 3, 10 and 80 μg. 30,000 counts per minute (cpm) of radiolabeled NRE probe or radiolabeled NRE shuffle probe are used for each condition. After incubation, the complexes are resolved in a non-denaturing polyacrylamide gel. The result of the migration is read by detecting the radioactivity emitted by the probes.

Results

Study of the Expression of the Short Messenger NXNL1 and the Long Messenger NXNL1

In Macaque

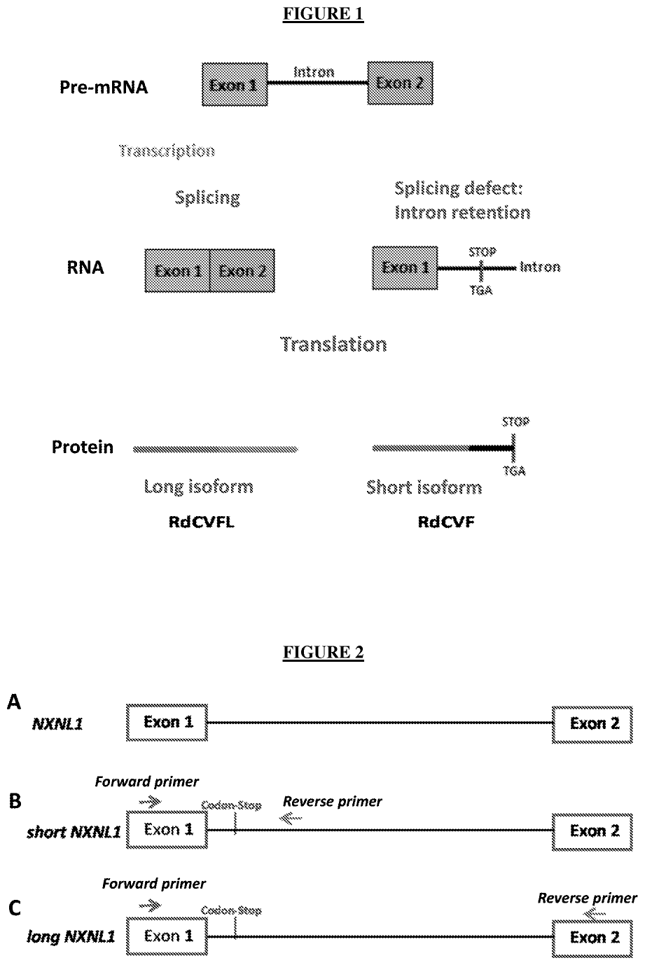

The expression of short and long messengers from the NXNL1 gene in primate was studied by RT-PCR. The forward primer is used for both messengers and was therefore chosen in the part of the NXNL1 gene common to both messengers, exon 1, whereas the reverse primer amplifying the messenger coding for RdCVF was chosen in the intron, located in 5′ from the stop codon in phase. The reverse primer for the messenger coding for RdCVFL was selected in exon 2 (FIG. 2).

The expression was studied in the different part of the retina obtained by dissection. The fovea represents an area enriched in cones, responsible for the central vision. It appears as a small depression and is located in the center of the macula. The macula, which is a region characterized by a yellowish patch due to a yellow xanthophyll pigment is highly specialized and only populated with cones, has also been isolated. It is located between 0.5 and 1 mm around the fovea and corresponds with the latter to the region of the retina called the central retina. The rods are present only from 1 mm from the center of the fovea, in the region called peripheral retina which, as opposed to the macula, has a much lower cone density. This region of the retina is generally divided into 4 zones. The near periphery, the medium periphery, the distant periphery and the ora serrata or extreme periphery, not studied here. Specifically, the rod density is zero in the fovea, maximum at an eccentricity of 5 to 7 mm, and falls slowly around the periphery. In order to analyze and compare the difference expression of a gene or a protein in macaque rods and cones, the near peripheral retina extending 1.5 mm from the macula was isolated, and the medium peripheral retina measuring 3 mm from the nearby peripheral retina and finally the distant peripheral retina which can extend between 9 to 15 mm since the macula.

The results of RT-PCR indicate that short messenger NXNL1 is approximately 20-fold more expressed in the near and distant peripheral retina than in the macula, and at least 30-fold more expressed in the medium peripheral retina than in the macula (FIG. 3A). The long messenger NXNL1 is approximately 3.5 times more expressed in the near peripheral retina than in the macula, 10 times more expressed in the medium peripheral retina than in the macula, and about 7 times more expressed in the distant peripheral retina than in the macula (FIG. 3B).

The overall results indicate that the difference of expression of the short messenger NXNL1 between the rods and cones of the macaque retina is greater than that of the long messenger NXNL1. These data indicate that the macula expresses the short messenger of NXNL1 20 to 30 times less than the peripheral retina whereas the long messenger of NXNL1 is approximately 3 to 10 times less expressed in the macaque macula than in the peripheral retina. It seems that the macaque cones do not express or at a very low level, the short messenger of NXNL1, while the macaque rods express the two messengers of NXNL1.

In Human

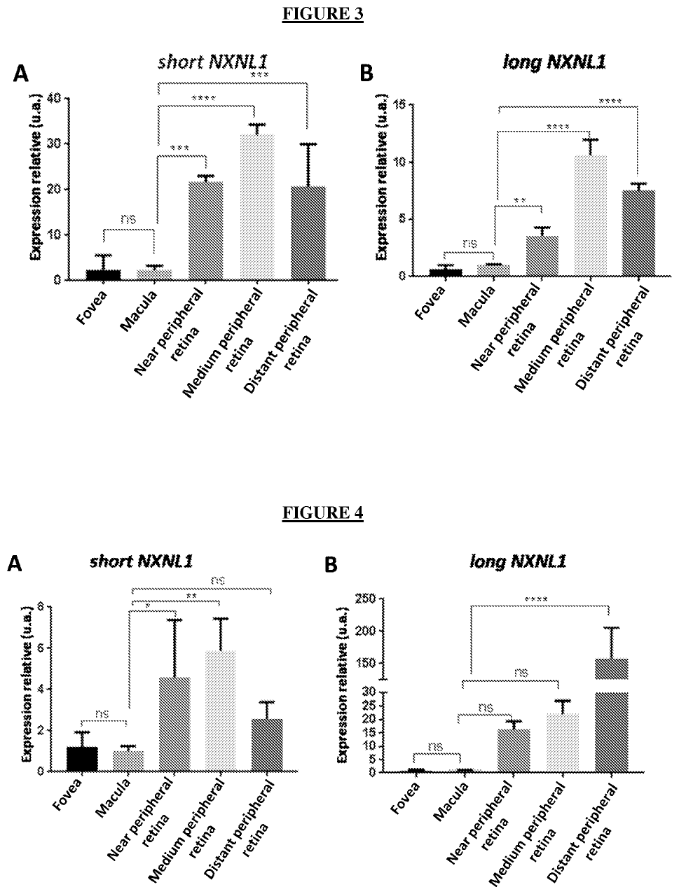

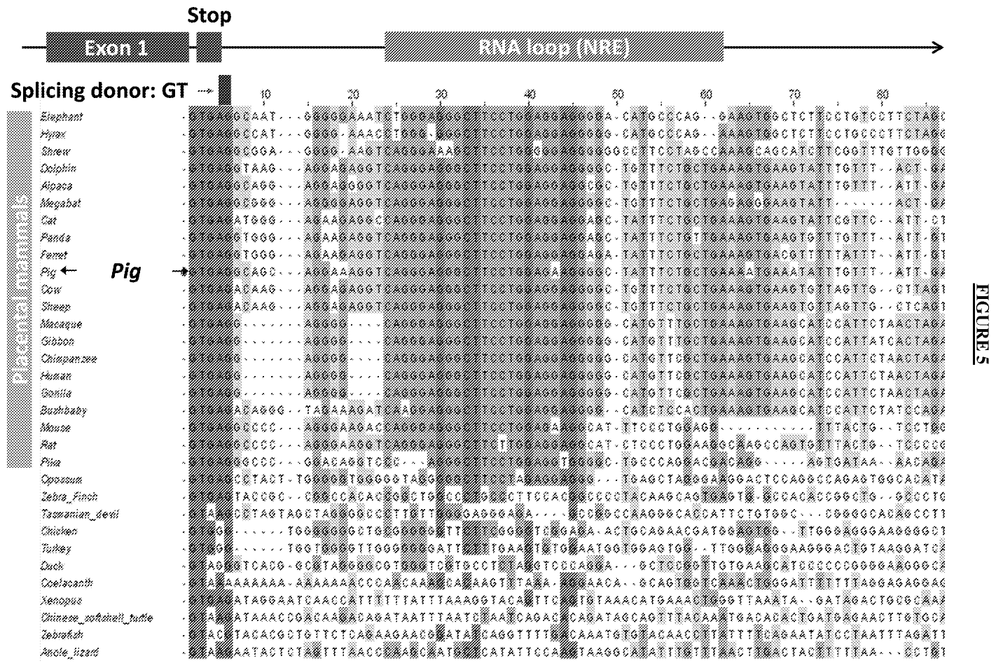

These experiments were also carried out from human retinas. As with the macaque retina, fovea, macula, near, medium and distant peripheral retina were isolated from human retinas and the RNAs extracted and analyzed (FIGS. 4A and 4B).

These results showed that in human, the short and the long messenger NXNL1 are expressed at a very low level in cones, compared to the rods which express at a high level the long messenger of NXNL1 that codes for RdCVFL and the short messenger of NXNL1 that codes for RdCVF. Taking into account the y-axis of FIGS. 3 and 4, the short messenger NXNL1 is less expressed in humans than in the macaque and the opposite is observed for the expression of the long messenger NXNL1.

The results of these experiments confirm the results obtained from the macaque retina, i.e., the short messenger NXNL1 is significantly more expressed in the peripheral retina than in the human central retina (fovea/macula).

Conclusion: In macaque and human retinas, cones which are located in fovea and macula, slightly express the short messenger NXNL1 encoding the isoform RdCVF compared to the others parts of the peripheral retina mainly composed by rods, which both expressed the long and the short messenger NXNL1.

Involvement of Nucleolin in Alternative Splicing of NXNL1 Gene

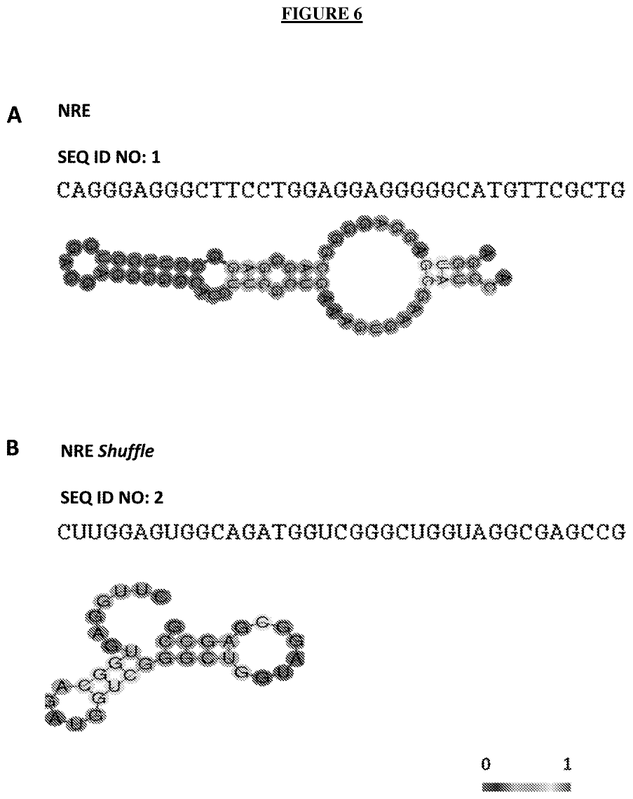

The Inventors highlighted that the RNA sequence of NXNL1 gene on the pre-RNA adopts a conserved hairpin secondary structure named nucleolin responsive element (NRE) (FIG. 6). This NRE sequence is phylogenetically well-conserved through mammals (FIG. 5).

Surprisingly, they discovered that nucleolin (NCL) binds specifically to the NRE.

An oligonucleotide probe was synthesized from the NRE RNA sequence to which cytochrome Cyanine 3 (Cy3) was added in 3′. The sequence named NRE shuffle, corresponding to a mutated NRE sequence with the mutated nucleotides arranged to destabilize the secondary structure of the NRE was also synthesized with a 3′ Cy3 and used as a negative control of the experiments (Table 2).

TABLE 2
Sequence of NRE and NRE Shuffle

| | |
|---|---|
| Sequence | SEQ ID NO: 1 |
| NRE | CAGGGAGGGCUUCCUGGAGGAGGGGGCAUGUUCGCUG |
| Sequence | SEQ ID NO: 2 |
| NRE | CUUGGAGUGGCAGAUGGUCGGGCUGGUAGGCGAGCCG |
| shuffle | |

These probes were incubated on the HuProt™ version 2.0 microarray chip with 19,951 distinct proteins on its surface to identify proteins that could interact specifically with NRE. All the purified recombinant proteins present on this chip are coupled to an N-terminal glutathione-S-transferase (GST) and labeled with a poly histidine tag (His6-tag). These proteins are grafted in duplicate on a glass slide previously coated with a polymer having different functions for coupling with GST proteins such as aldehyde, epoxy, carboxyl or hydroxyl functions.

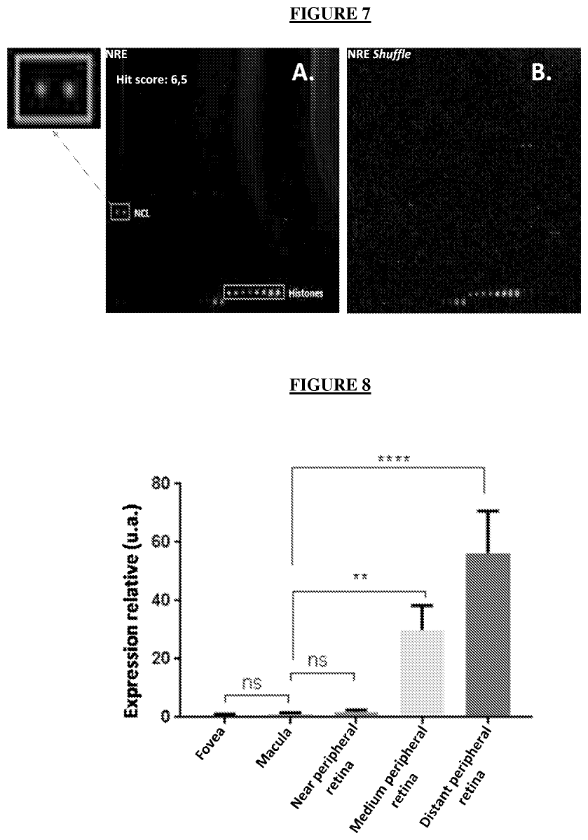

These grafted proteins are produced in the yeast S. cerevisiae. Proteins used as negative controls such as GST, bovine serum albumin (BSA), histones or immunoglobulins are also present on the chip. These proteins which interacted with the probes are ranked according to a score (or hit) that is correlated to the fluorescence intensity emitted as a result of the interaction between the protein and the RNA probe. The stronger is the interaction, the higher is the intensity of fluorescence and the higher is the score. The results of this experiment indicate that the nucleolin specifically interacts with the NRE sequence (FIG. 7).

From the RNAs extracted from the fovea, the macula and from the 3 regions of the macaque peripheral retina, a quantitative RT-PCR using specific primers amplifying the gene which codes for NCL has been carried out. NCL messenger expression normalized by the gene that codes for GAPDH is greater in the medium and distant peripheral retina than in the near peripheral retina or in the macula and in the fovea where the cones are (FIG. 8).

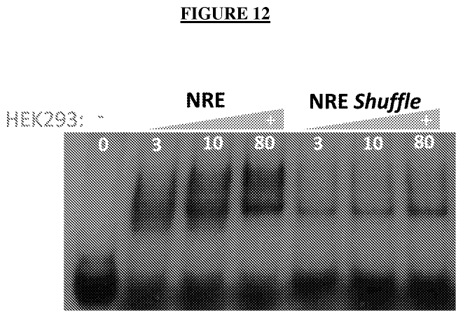

From another macaque retina, the different regions of the retina are removed as previously described to obtain a section of the fovea/macula, the near, medium and distant peripheral retina. From these sections, the protein lysates were extracted and a western blot made (FIG. 11). The different regions of the retina are controlled and it is observed that in the fovea/macula, mainly composed of cones, the human cone transducin protein (GNAT2) is more expressed than in the other more distant regions of the retinal center. In contrast, rhodopsin, a rod-specific protein, is expressed on the periphery of the macaque retina and is not present at all in the center of the retina (macula) where the rods are completely absent. These results validate the sections by showing that the cones are rather localized in the macaque retina fovea/macula while the rods are rather present in the peripheral retina.

At the same time, the expression of the NXNL1 short messenger encoding RdCVF was followed in each of the isolated sections of the retina.

The results of the western blot and the RT-PCR show a correlation in the presence of the NCL protein, and the high level of the mRNA coding for the short isoform RdCVF in retina sections mainly comprising rods. On the contrary, in the fovea/macula where there are only cones, NCL is not detected and the mRNA coding for the short isoform RdCVF is not significantly expressed (FIGS. 11B and 11C).

NCL is known to migrate aberrantly on an electrophoresis gel in presence of denaturing agents. Indeed, NCL can either migrate to its theoretical size expected 100 kDa, or at a smaller size of 60 kDa. Assumptions about the existence and presence of different phosphorylation sites or cleavages in this protein are advanced to justify this difference in migration. Some work explains that NCL which has many phosphorylation sites can be partially cleaved generating different NCL proteins of different sizes, full-length NCL or NCL-fragment (Gotzmann et al., 1997). This protein, depending on its size, is either localized in the nucleus, compartment where the pre-RNA is treated and spliced or in the cytoplasm. It is known that the full-length NCL is found in the nucleus while the NCL fragment is found in the cytoplasm (Billing et al., 2005; Gotzmann et al., 1997). Western blot results using NCL-specific antibody indicate that the more we go away from the center of the retina, the more the longer the NCL full-length is expressed. At the opposite, the more we move away from the center of the retina, and the less the NCL fragment is expressed. However, both forms are more expressed at the periphery of the retina than in the center (fovea/macula). Interestingly, it seems that the full-length form, nuclear, is more present in the peripheral retina where rods are located, photoreceptors cells where the retention of the intron takes place. Quantification of western blot band intensities of different forms of NCL indicates that the full-length NCL is 9-fold more expressed in the medium peripheral retina than in the macula and 8-fold more expressed in the distant peripheral retina than in the macula. These results also indicate that full-length NCL is 8-fold more expressed in the distant peripheral retina than the fragment NCL (FIG. 9).

The full-length NCL that is known to be localized in the nucleus, which corresponds to the compartment where the NXNL1 pre-mRNAs are presumably spliced, is more expressed in the retinal region where the rods are predominantly present. These results are consistent with the hypothesis that NCL could lead to the retention of the NXNL1 intron, thus allowing the expression of the short messenger NXNL1 which codes for RdCVF.

The binding between NCL and the NRE was confirmed by a gel-shift assay (FIG. 12).

The gel-shift assay shows NCL of the protein extract binds specifically to the NRE probe. In fact, previously to this assay, a western blot has been performed from a protein extract of HEK293 cells, using anti-NCL antibodies to identify the migration size of NCL in this condition. Thus, it was possible to identify on the gel, that the observed bands correspond to the radioactive probe bound to the NCL protein, the probe being retained on the gel because of its binding to a protein migrating to the size of NCL.

The gel results also show that NCL binds specifically to the NRE of SEQ ID NO: 1. In fact, NRE shuffle (SEQ ID NO: 2) which corresponds to the NRE sequence with some mutations, binds weakly the protein.

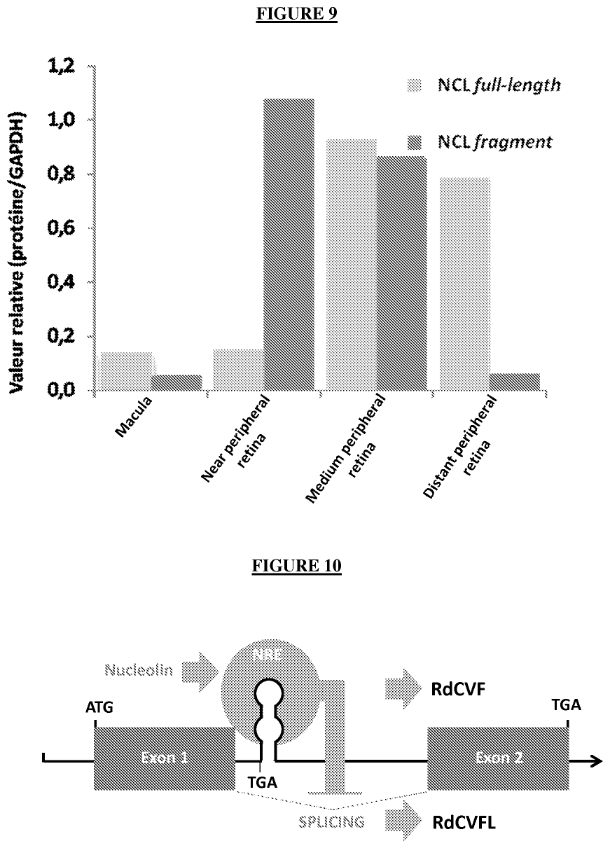

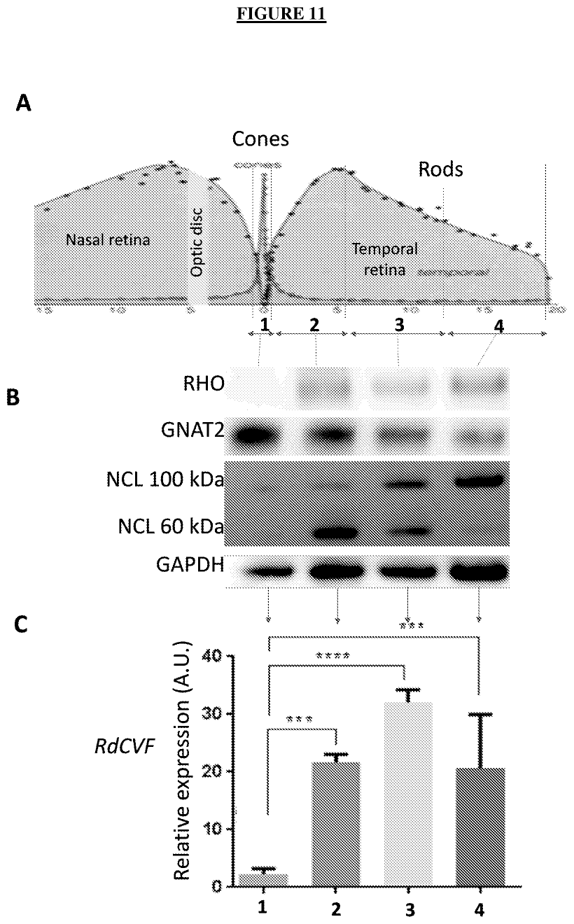

All the results of the present application demonstrate that the binding of NCL on the NRE sequence leads to the intron retention and thus to the production of the short isoform RdCVF in rods (FIG. 10). However, this alternative splicing does not seem to occur in cones, but only in rods.

Method of Delivery of Nucleolin in Cones by AAV Vector

As explained before RdCVF acts by binding to the cell-surface complex BSG1/GLUT1 on the cones membrane. This stimulates the glucose uptake by cones and thus promotes retinal cone survival.

In order to improve cones survival, for example in case of the degeneration of rods and thus in case of a decrease of RdCVF production by rods, the Inventors have thus proposed to force the expression of NCL in cones by delivering into retina, an AAV vector expressing NCL (AAV-NCL) by subretinal injection. The expression of NCL will lead to the intron retention in the pre-RNA of NXNL1 gene in cones, and thus to the expression of the short messenger coding for RdCVF in cones.

AAV vectors carrying cDNA encoding NCL (accession number on NCBI GeneID 4691) are produced by the plasmid co-transfection method (Grieger et al., 2006). Recombinant AAV is purified by cesium chloride or iodixanol gradient ultracentrifugation. The viral eluent is buffer exchanged and concentrated with Amicon ultra-15 centrifugal filter units in phosphate buffer saline (PBS) and titrated by quantitative PCR relative to a standard curve.

### REFERENCES

Ait-Ali, N., Fridlich, R., Millet-Puel, G., Clerin, E., Delalande, F., Jaillard, C., Blond, F., Perrocheau, L., Reichman, S., Byrne, L. C., Olivier-Bandini, A., Bellalou, J., Moyse, E., Bouillaud, F., Nicol, X., Dalkara, D., van Dorsselaer, A., Sahel, J. A., and Léveillard, T. Rod-derived cone viability factor promotes cone survival by stimulating aerobic glycolysis. Cell 2015, 161, 817-832.
Brennan L A, Lee W, Kantorow M. TXNL6 is a novel oxidative stress-induced reducing system for methionine sulfoxide reductase a repair of a-crystallin and cytochrome C in the eye lens. PLoS ONE. 2010 Nov. 4; 5 (11).
Buch H, Vinding T, La Cour M, Appleyard M, Jensen G B, Nielsen N V. Prevalence and causes of visual impairment and blindness among 9980 Scandinavian adults: the Copenhagen City Eye Study. Ophthalmology. 2004; 11 1(1):53-61.
Byrne L C, Luna G, Fisher S K, Clérin E, Sahel J A, Léveillard T, Flannery J G. Viral-mediated RdCVF and RdCVFL expression protects cone and rod photoreceptors in retinal degeneration. J Clin Invest, 2015 February, 125(1):105-16.
Decembrini, S., Martin, C., Sennlaub, F., Chemtob, S., Biel, M., Samardzija, M., Moulin, A., Behar-Cohen, F., and Arsenijevic, Y. Cone Genesis Tracing by the Chrnb4-EGFP Mouse Line: Evidences of Cellular Material Fusion after Cone Precursor Transplantation. Molecular therapy: the journal of the American Society of Gene Therapy. 2017; 25, 634-653.
Elachouri G, Lee-Rivera I, Clerin E, Argentini M, Fridlich R, Blond F, Ferracane V, Yang Y, Raffelsberger W, Wan J, Bennett J, Sahel J A, Zack D J, Léveillard T. Thioredoxin rod-derived cone viability factor protects against photooxidative retinal damage. Free radical biology & medicine 2015, 81:22-29.
Fahling, M., Steege, A., Perlewitz, A., Nafz, B., Mrowka, R., Persson, P. B., and Thiele, B. J. Role of nucleolin in posttranscriptional control of MMP-9 expression. Biochimica et biophysica acta. 2005; 1731, 32-40.
Funato Y, Miki H. Nucleoredoxin, a Novel Thioredoxin Family Member Involved in Cell Growth and Differentiation. Antioxid Redox Signal. 2007; 9(8): 1035-1058.
Gotzmann, J., Eger, A., Meissner, M., Grimm, R., Gerner, C., Sauermann, G., and Foisner, R. Two-dimensional electrophoresis reveals a nuclear matrix-associated nucleolin complex of basic isoelectric point. Electrophoresis. 1997; 18, 2645-2653.
Grieger J C, Choi V W, Samulski R J. Production and characterization of adeno-associated viral vectors. Nat Protoc. 2006; 1(3):1412-1428.
Léveillard T, Mohand-Saïd S, Lorentz O, Hicks D, Fintz A C, Clérin E, Simonutti M, Forster V, Cavusoglu N, Chalmel F, Dollé P, Poch O, Lambrou G, Sahel J A. Identification and characterization of rod-derived cone viability factor. Nat Genet. 2004; 36(7):755-759.
Léveillard, T., and Ait-Ali, N. Cell Signaling with Extracellular Thioredoxin and Thioredoxin-Like Proteins: Insight into Their Mechanisms of Action. Oxidative medicine and cellular longevity. 2017: 8475125.
Lillig C H, Holmgren A. Thioredoxin and Related Molecules-From Biology to Health and Disease. Antioxid Redox Signal. 2007; 9(1):25-47.
Mei X., Chaffiol, A., Kole, C., Yang Y., Millet-Puel G., Clérin E., Aït-Ali N., Bennett, J., Dalkara D., Sahel J A, Duebel, J., Léveillard T. The thioredoxin encoded by the Rod-derived Cone Viability Factor gene protects cone photoreceptors against oxidative stress. Antioxid Redox Signal. 2016 Jun. 1; 24(16):909-23.
Salvetti, A., Coute, Y., Epstein, A., Arata, L., Kraut, A., Navratil, V., Bouvet, P., and Greco, A. Nuclear Functions of Nucleolin through Global Proteomics and Interactomic Approaches. Journal of proteome research. 2016; 15, 1659-1669.
Shin, S. H., Lee, G. Y., Lee, M., Kang, J., Shin, H. W., Chun, Y. S., and Park, J. W. Aberrant expression of CITED2 promotes prostate cancer metastasis by activating the nucleolin-AKT pathway. Nature communications. 2018 Oct. 5; 9(1):4113.
Yang Y, Mohand-Said S, Danan A, Simonutti M, Fontaine V, Clerin E, Picaud S, Léveillard T, Sahel J A. Functional Cone Rescue by RdCVF Protein in a Dominant Model of Retinitis Pigmentosa. Mol Ther. 2009; 17(5):787-795.

1. A method for treating a patient suffering from a retinal degenerative disease comprising administering to said patient a therapeutically effective amount of nucleolin polynucleotide or polypeptide.

2. The method according to claim 1, wherein said retinal degenerative disease is selected from the group consisting of: retinitis pigmentosa, age-related macular degeneration, Bardet-Biedel syndrome, Bassen-Kornzweig syndrome, Best disease, choroidema, gyrate atrophy, Leber congenital amaurosis, Refsum disease, Stargardt disease and Usher syndrome.

3. The method according to claim 1, wherein said retinal degeneration disease is retinitis pigmentosa.

4. The method for use according to claim 1, wherein the nucleolin polynucleotide is comprised in an expression vector.

5. The method according to claim 4, wherein said expression vector is a plasmid or viral particle.

6. The method according to claim 4, wherein said expression vector is an adeno-associated vector.

7. The method according to claim 1, wherein the nucleolin polynucleotide or polypeptide is administered by sub-retinal injection or intravitreal injection.

8. The method according to claim 1, wherein the nucleolin polynucleotide or polypeptide is formulated in a pharmaceutically acceptable ophthalmic vehicle.

9. The method according to claim 1, wherein the nucleolin polynucleotide or polypeptide is administered by intravascular injection.

10. The method according to claim 1, wherein the patient has undergone transplantation of cones into the retina.

11. The method according to claim 10, wherein said cones are induced pluripotent stem cell-derived cones (iPSC-cones).

12. A method of treating a retinal degenerative disease in a patient in need thereof, comprising
administering to the patient a recombinant cone overexpressing nucleolin.

13. The method according to claim 12, wherein the recombinant cone overexpressing nucleolin is transplanted into the patient retina.

14. Recombinant cone overexpressing nucleolin.

15. The method according to claim 1, wherein the nucleolin is human nucleolin.

16. The method according to claim 12, wherein the recombinant cone is a recombinant iPSC-cone.

17. The method according to claim 12, wherein the nucleolin is human nucleolin.

18. The recombinant cone overexpressing nucleolin according to claim 14 wherein the nucleolin is human nucleolin.


SEQUENCE LISTING (7 sequences)

SEQ ID NO: 1 - RNA, 37 nt, artificial sequence
  Synthetic NRE mRNA sequence
cagggagggcuuccuggaggagggggcauguucgcug

SEQ ID NO: 2 - RNA, 37 nt, artificial sequence
  Synthetic NRE shuffle
cuuggaguggcagauggucgggcugguaggcgagccg

SEQ ID NO: 3 - DNA, 20 bp, artificial sequence
  Synthetic Primer hRdCVF F Exon 1
gagttctatgtactgcgggc

SEQ ID NO: 4 - DNA, 21 bp, artificial sequence
  Synthetic Primer hRdCVF R Intron 1
cttcactttcagcgaacatgc

SEQ ID NO: 5 - DNA, 18 bp, artificial sequence
  Synthetic Primer hRdCVF R Exon 2
tccactgagaactggcgc

SEQ ID NO: 6 - DNA, 20 bp, artificial sequence
  Synthetic Primer hNCL 1 F
gcgttggaactcactggttt

SEQ ID NO: 7 - DNA, 20 bp, artificial sequence
  Synthetic Primer hNCL 1 R
ccgcagcatcttcaaacact